(19) United States
(12) Patent Application Publication
(10) Pub. No.: US 2024/0294448 A1
(43) Pub. Date: Sep. 5, 2024

(54) HYDROGENATION OF ESTERS TO ALCOHOLS IN THE PRESENCE OF AN MN-PNN COMPLEX

(71) Applicant: BASF SE, Ludwigshafen am Rhein (DE)

(72) Inventors: Thomas SCHAUB, Ludwigshafen am Rhein (DE); Mathias SCHELWIES, Ludwigshafen am Rhein (DE); Viktoriia ZUBAR, Heidelberg (DE)

(21) Appl. No.: 18/571,768

(22) PCT Filed: Jun. 10, 2022
(86) PCT No.: PCT/EP2022/065787

§ 371 Date: Dec. 19, 2023

(30) Foreign Application Priority Data
Jun. 21, 2021 (EP) 21180529.6

(51) Int. Cl. C07C 29/149; B01J 31/18; C07D 307/92

(52) U.S. Cl. CPC C07C 29/149; B01J 31/189; C07D 307/92

(57) ABSTRACT

A method for hydrogenating an ester of the general formula (III)

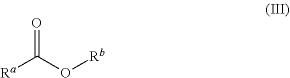

with molecular hydrogen to give the alcohols

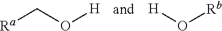

at a temperature of 50 to 200° C. and a pressure of 0.1 to 20 MPa abs in the presence of a manganese(I) complex, in which the manganese complex comprises a tridentate ligand L with the general formula (II)

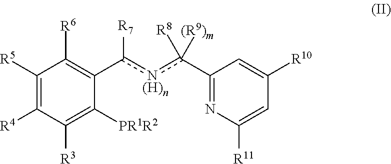

and comprises at least two carbonyl ligands.

The present invention relates to a method for hydrogenating an ester with molecular hydrogen to give the corresponding alcohols in the presence of a manganese complex having a tridentate PNN ligand.

Alcohols are not only important solvents, they are also important intermediates and synthetic units, for example for the production of pharmaceuticals, plant protection agents or fragrances. Depending on the type of alcohol desired and the availability of the corresponding starting material, direct hydrogenation of the corresponding ester with hydrogen or reduction with reducing agents are often the methods of choice.

The synthesis of alcohols from esters is usually effected by using metal hydrides such as LiAlH₄ or NaBH₄, by heterogeneous catalytic hydrogenation with hydrogen, or by homogeneous catalytic hydrogenation with hydrogen. Homogeneous catalytic hydrogenations with hydrogen often allow less drastic reaction conditions with better selectivity at the same time. In particular, the use of ruthenium complexes having polydentate phosphorus-, sulfur- and nitrogen-containing ligands has proved successful in this respect according to the prior art, but in recent years alternatives have also been developed that comprise manganese as the active metal.

For instance Beller et al. describe in Angewandte Chemie International Edition 2016, Vol. 55, pages 15364-15368 the use of Mn complexes having a so-called pincer ligand of the PNP type for the hydrogenation of esters to alcohols. Ligands are used here which have an N H unit in the center, with two ethyldialkylphosphine units attached thereto. The manganese atom is present in the oxidation state +I and also bears at least two carbonyl ligands. With these catalysts, a series of esters and lactones can be hydrogenated to the corresponding alcohols or diols.

Disadvantages of these catalysts are the relatively high catalyst loadings of 2 mol % and the 10 mol % of KOtBu as the necessary co-catalyst which must be used in order to obtain high conversions in the hydrogenation.

Milstein et al. in Chemistry, a European Journal 2017, Vol. 23, pages 5934-5938 describe the use of Mn complexes having a so-called pincer ligand of the PNN type for hydrogenation of esters to alcohols. The tridentate pincer ligand described has a pyridyl group as backbone and, as donor groups, a phosphino and NHR group with an alkyl group. With these catalysts, a series of esters and lactones can be hydrogenated to the corresponding alcohols or diols.

The disadvantage of using the pincer ligands of the PNN type mentioned is their complex, multistage synthesis, using challenging reagents such as n-butyllithium, starting from 2,6-dimethylpyridine. Disadvantages of these catalysts are also the relatively high catalyst loadings of at least 1 mol % and the use of expensive and difficult to handle KH as necessary co-catalyst, which have to be used to obtain high conversions in hydrogenation. With the simpler and cheaper alkoxide bases, only low conversions can be achieved with this manganese catalyst.

Pidko et al. in Angewandte Chemie International Edition 2017, Vol. 56, pages 7531-7534, describe the use of Mn complexes with simple bidentate PN ligands for the hydrogenation of esters to alcohols. The ligands used here have an NH₂ unit, an ethylene bridge and a PR₂ (R=alkyl, aryl) group. The manganese atom is present in the oxidation state +I and also bears at least two carbonyl ligands. The advantage here is that the ligands are easy to produce.

However, only the hydrogenation of acyclic esters and not lactones using these catalysts is described. Further disadvantages of these catalysts are the relatively high catalyst loadings of 1 mol % and the very high loadings required of 10-75 mol % of KOtBu as a necessary co-catalyst, which have to be used in order to obtain high conversions in hydrogenation. Conversions of the ester above 90% can be achieved here only with base loadings of at least 50 mol %, which is disadvantageous for an economic process.

Clark et al. in Organic Letters 2018, Vol. 20, pages 2654-2658 describe the use of Mn complexes having a so-called pincer ligand of the PNN type for the hydrogenation of esters to alcohols. The tridentate pincer ligand described has an NH group as backbone and a pyridyl group and a PPh2 group bridged viaferrocene as donor groups. The manganese atom is present in the oxidation state +I and also bears three carbonyl ligands. The advantage here is that the ligands are easy to produce. With this catalyst, a series of esters and lactones can be hydrogenated to the corresponding alcohols or diols.

The advantage of this catalyst is the low catalyst loadings of only 0.1 mol %, which are necessary, for example, to hydrogenate the lactone sclareolide to the corresponding diol at moderate yields of 75%.

The disadvantage of using these pincer ligands of the PNN type is their very complex, multistage synthesis, using challenging reagents such as n-butyllithium.

Another disadvantage of this catalyst is the relatively high required loading of 10 mol % base as co-catalysts, as with the lactone sclareolide, in order to achieve good conversions.

WO 2021/001240 A1 describes the use of easily prepared PNN ligands, with which highly active ruthenium catalysts can be produced for the hydrogenation of esters, in which only very little base is required as a co-catalyst or where the co-catalyst can also be active without addition of base. However, nothing was known about their suitability as ligands for active manganese catalysts until now and structurally it differs from the ligands for manganese catalysts described above in ester hydrogenation, which is why it was not expected that highly active Mn catalysts could be produced with this ligand.

The object of the present invention was to find a method for homogeneously catalyzed hydrogenation of esters to the corresponding alcohols which does not have the stated disadvantages of the prior art or only to a minor extent, is easy to carry out with regard to the apparatus required and reaction conditions and which enables the highest possible space-time yield.

In particular, the catalytically active complex should be directly preparable from readily available feedstocks, have high activity in the hydrogenation of esters to alcohols and require as little cocatalyst as possible and ultimately be disposable without undue effort. In this context, above all the complex-forming ligand is of particular importance, which is as simple as possible to produce, but still generates a manganese catalyst with high activity.

Surprisingly, a method has been found for hydrogenating an ester of the general formula (III)

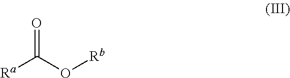

in which the radicals R^{a} and R^{b} are each independently a carbon-containing organic, linear or branched, non-cyclic or cyclic, saturated or unsaturated, aliphatic, aromatic or araliphatic radical which is unsubstituted or interrupted or substituted by heteroatoms or functional groups and has a molar mass of 15 to 10 000 g/mol, wherein the two radicals R^{a} and R^{b} may also be bonded to each other,
with molecular hydrogen to give the alcohols

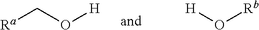

at a temperature of 50 to 200° C. and a pressure of 0.1 to 20 MPa abs in the presence of a manganese(I) complex (I), in which the manganese complex comprises a tridentate ligand L of the general formula (II)

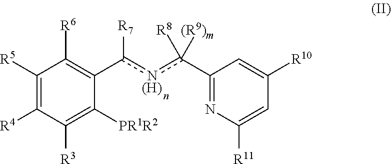

and comprises at least two carbonyl ligands, wherein
R¹, R² are each independently an aliphatic hydrocarbon radical having 1 to 8 carbon atoms, an aromatic hydrocarbon radical having 6 or 10 carbon atoms or an araliphatic hydrocarbon radical having 7 to 12 carbon atoms, where the hydrocarbon radicals specified are unsubstituted or substituted by 1 to 3 methoxy, thiomethoxy or dimethylamino groups, and the two radicals R¹ and R² may be bonded to each other to form a 5- to 10-membered ring including the phosphorus atom,
R³, R⁴, R⁵, R⁶, R¹⁰, R¹¹ are each independently hydrogen, linear C₁ to C₄-alkyl, branched C₃ to C₄-alkyl, methoxy, hydroxyl, trifluoromethyl, nitrile or dialkylamino each independently having 1 to 4 carbon atoms per alkyl group,
R⁷, R⁸, R⁹ are each independently hydrogen, linear C₁ to C₄-alkyl or branched C₃ to C₄-alkyl,
n, m are each independently 0 or 1, and
the solid-dashed double lines are a single or double bond, with the proviso that in the case of n=1, both solid-dashed double lines are a single bond
   and m is 1, and
in the case of n=0, one solid-dashed double line represents a single bond and the other solid-dashed double line represents a double bond, wherein in the case of a double bond on the side facing the phenyl ring m=1, in the case of a double bond on the side facing the pyridyl ring m=0, or both solid-dashed double lines represent a single bond and m
   equals 1.

The core of the method according to the invention is the use of a manganese(I) complex, which comprises a tridentate ligand L of the general formula (II) and at least two carbonyl ligands, in the hydrogenation of esters with molecular hydrogen to give the corresponding alcohols.

The tridentate ligand L is a so-called PNN ligand of the general formula (II)

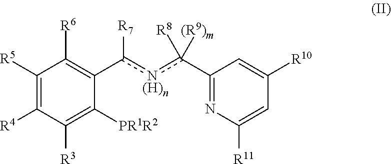

in which
R¹, R² are each independently an aliphatic hydrocarbon radical having 1 to 8 carbon atoms, an aromatic hydrocarbon radical having 6 or 10 carbon atoms or an araliphatic hydrocarbon radical having 7 to 12 carbon atoms, where the hydrocarbon radicals specified are unsubstituted or substituted by 1 to 3 methoxy, thiomethoxy or dimethylamino groups, and the two radicals R¹ and R² may be bonded to each other to form a 5- to 10-membered ring including the phosphorus atom,
R³, R⁴, R⁵, R⁵, R¹⁰, R¹¹ are each independently hydrogen, linear C₁ to C₄-alkyl, branched C₃ to C₄-alkyl, methoxy, hydroxyl, trifluoromethyl, nitrile or dialkylamino each independently having 1 to 4 carbon atoms per alkyl group,
R⁷, R⁸, R⁹ are each independently hydrogen, linear C₁ to C₄-alkyl or branched C₃ to C₄-alkyl,
n, m are each independently 0 or 1, and
the solid-dashed double lines are a single or double bond, with the proviso that in the case of n=1, both solid-dashed double lines are a single bond
   and m is 1, and
in the case of n=0, one solid-dashed double line represents a single bond and the other solid-dashed double line represents a double bond, wherein in the case of a double bond o n the side facing the phenyl ring m=1, in the case of a double bond on the side facing the pyridyl ring m=0, or both solid-dashed double lines represent a single bond and m=1.

Tridentate means that ligand L (II) occupies three coordination sites in the manganese(I) complex (I). The three ligand donor atoms are the P and the two N atoms, from which the name PNN ligand is derived.

With respect to the environment of the central donor atom, the ligand can in principle have four different substructures, which are explained in more detail below.

(1) In the case of n=1, both solid-dashed double lines represent a single bond and m is 1. This results in the general formula (IIa). Ligand (IIa) is neutral, so it has a charge of “0”.

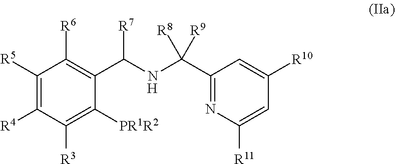

In the case of n=0, there are a total of three different substructures.

(2) If n=0 and the solid-dashed double line facing the phenyl ring is a double bond and the solid-dashed double line facing the pyridyl ring is a single bond, then m is equal to 1. This results in the general formula (IIb). Ligand (IIb) is neutral, so it has a charge of “0”.

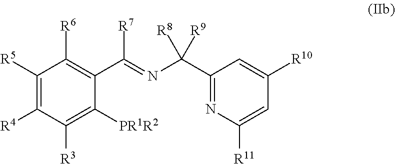

(3) If n=0 and the solid-dashed double line facing the pyridyl ring is a double bond and the solid-dashed double line facing the phenyl ring is a single bond, then m is equal to 0. This results in the general formula (IIc). Ligand (IIc) is neutral, so it has a charge of “0”.

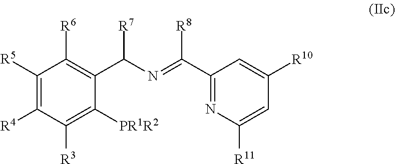

(4) In the fourth variant, n=0 also, but both solid-dashed double lines are single bonds and m is 1. The N atom thus has a negative charge. This results in the general formula (IId). The ligand (IId) thus has a charge of “−1”.

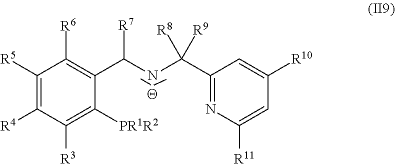

The radicals R¹ and R² of the ligand (II) may vary to a wide extent and are each independently an aliphatic hydrocarbon radical having 1 to 8 carbon atoms, an aromatic hydrocarbon radical having 6 or 10 carbon atoms or an araliphatic hydrocarbon radical having 7 to 12 carbon atoms, where the hydrocarbon radicals specified may be unsubstituted or substituted by 1 to 3 methoxy, thiomethoxy or dimethylamino groups, and the two radicals R¹ and R² may also be bonded to each other to form a5- to 10-membered ring including the phosphorus atom.

In the case of an aliphatic hydrocarbon radical, this may be unbranched or branched or linear or cyclic. The aliphatic hydrocarbon radical preferably has 1 to 6 carbon atoms, particularly preferably 1 to 4 carbon atoms and especially preferably 1 to 2 carbon atoms. Specific examples include methyl, ethyl, isopropyl, n-propyl, n-butyl, isobutyl, tert-butyl (also referred to as tBu) and cyclohexyl (also referred to as Cy).

In the case of an aromatic hydrocarbon radical, this is phenyl (also referred to as Ph), 1-naphthyl or 2-naphthyl.

Araliphatic hydrocarbon radicals comprise aromatic and aliphatic elements, regardless of whether these are bonded to the phosphorus atom in the ligand L via an aliphatic or an aromatic group. The araliphatic hydrocarbon radicals preferably have 7 to 10 carbon atoms and particularly preferably 7 to 9 carbon atoms. Specific examples include o-tolyl, m-tolyl, p-tolyl and benzyl.

In the case of a ring including the phosphorus atom, it is preferably a ring having 5 to 6 atoms, including the phosphorus atom. Examples include butane-1,4-diyl, pentane-1,5-diyl and 2,4-dimethylpentane-1,5-diyl.

The aliphatic, aromatic and araliphatic hydrocarbon radicals mentioned, which may also be bonded to one another to form a ring including the phosphorus atom, may be unsubstituted or substituted by 1 to 3 methoxy, thiomethoxy or dimethylamino groups. The number of carbon atoms of the individual hydrocarbon radicals specified above is to be understood as including the carbon atoms of the methoxy, thiomethoxy or dimethylamino groups. Specific examples include 3,5-dimethylphenyl, 3,5-dimethyl-4-methoxyphenyl, 3,5-dimethyl-4-thiomethoxyphenyl and 3,5-dimethyl-4-(dimethylamino)phenyl.

The radicals R¹ and R² are particularly preferably phenyl, p-tolyl, o-tolyl, 4-methoxyphenyl, 2-methoxyphenyl, cyclohexyl, isobutyl, tert-butyl, 3,5-dimethyl-4-methoxyphenyl, 3,5-tert-butyl-4-methoxyphenyl and 3,5-dimethylphenyl and especially preferably phenyl, p-tolyl, 3,5-dimethyl-4-methoxyphenyl, isobutyl and cyclohexyl, where preferably both radicals are the same.

The radicals R³, R⁴, R⁵, R⁶, R¹⁰ and R¹¹ are each independently hydrogen, linear C₁ to C₄-alkyl, branched C₃ to C₄-alkyl, methoxy, hydroxyl, trifluoromethyl, nitrile or dialkylamino each independently having 1 to 4 carbon atoms per alkyl group. Linear C₁ to C₄-alkyl include methyl, ethyl, n-propyl and n-butyl and branched C₃ to C₄-alkyl include isopropyl, sec-butyl and tert-butyl. Dialkylamino includes in particular amino radicals having identical alkyl groups, in particular dimethylamino, diethylamino, di-n-propylamino and di-n-butylamino.

The radicals R³ and R⁴ are preferably each independently hydrogen or methyl and particularly preferably hydrogen.

The radical R⁵ is preferably hydrogen, methyl, isopropyl, sec-butyl, tert-butyl, methoxy, hydroxyl or dialkylamino, particularly preferably hydrogen, methyl or hydroxyl and especially preferably hydrogen.

The radical R⁶ is preferably hydrogen.

The radical R¹⁰ is preferably hydrogen, methyl, isopropyl, sec-butyl, tert-butyl or methoxy, particularly preferably hydrogen, methyl or tert-butyl and especially preferably hydrogen.

The radical R¹¹ is preferably hydrogen, methyl, ethyl, methoxy, ethoxy or isopropyloxy and particularly preferably hydrogen, methyl or methoxy.

Especially preferred are ligands (II) in which
R³, R⁴, R⁵, R⁶, R¹⁰ and R¹¹ are hydrogen,
R³, R⁴, R⁵, R⁶ and R¹⁰ are hydrogen and R¹¹ is methyl,
R³, R⁴, R⁵, R⁶ and R¹⁰ are hydrogen and R¹¹ is methoxy,
R³, R⁴, R⁶, R¹⁰ and R¹¹ are hydrogen and R⁵ is methyl,
R³, R⁴, R⁶, R¹⁰ and R¹¹ are hydrogen and R⁵ is tert-butyl,
R³, R⁴, R⁵, R⁶ and R¹¹ are hydrogen and R¹⁰ is methyl,
R³, R⁴, R⁵, R⁶ and R¹¹ are hydrogen and R¹⁰ is tert-butyl, and
R³, R⁴, R⁵ and R⁶ are hydrogen and R¹⁰ and R¹¹ are methyl.

The radicals R⁷, R⁸ and R⁹ are each independently hydrogen, linear C₁ to C₄-alkyl or branched C₃ to C₄-alkyl. Linear C₁ to C₄-alkyl include methyl, ethyl, n-propyl and n-butyl and branched C₃ to C₄-alkyl include isopropyl, sec-butyl and tert-butyl.

The radicals R⁷, R⁸ and R⁹ are preferably each independently hydrogen, methyl, ethyl or n-propyl, particularly preferably hydrogen or methyl and especially preferably hydrogen.

Especially preferred are ligands (II) in which
R⁷, R⁸ and R⁹ are hydrogen,
R⁷ and R⁹ are hydrogen and R⁸ is methyl,
R⁷ is hydrogen and R⁸ and R⁹ are methyl,
R⁷ is methyl and R⁸ and R⁹ are hydrogen, and
R⁷ and R⁸ are methyl and R⁹ is hydrogen.

Particularly advantageous in the method according to the invention is the use of the ligands (II), in which
(i) n and m are in each case 1 and the two solid-dashed double lines represent a single bond (structure (IIa)), or
(ii) n is 0 and m is 1 and the solid-dashed double line facing the phenyl ring represents a double bond and the solid-dashed double line facing the pyridyl ring represents a single bond (structure (IIb)),
   and
   both radicals R¹ and R² are phenyl, p-tolyl, 3,5-dimethyl-4-methoxyphenyl, isobutyl or cyclohexyl,
   the radicals R³, R⁴ and R⁶ are hydrogen,
   the radicals R⁵ and R¹⁰ are hydrogen, methyl or tert-butyl,
   the radical R¹¹ is hydrogen, methyl or methoxy, and
   the radicals R⁷, R⁸ and R⁹ are hydrogen or methyl.

Accordingly, in the method according to the invention, the ligands L1, L2, L3, L4 and L5 are particularly suitable.

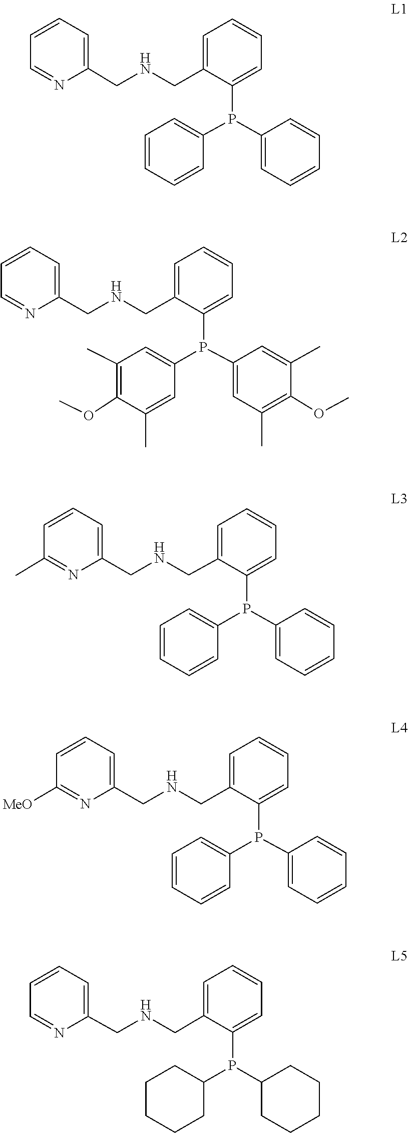

Ligand (II) can be obtained in a simple manner by condensation of a corresponding amine with a corresponding aldehyde or ketone (ligand (IIb) and (IIc)) and a possible subsequent reduction (ligand (IIa)) and a possible subsequent deprotonation under basic conditions (ligand (IId)).

There are in principle two different possibilities for the condensation. Firstly, it is possible to use 2-picolylamine or a corresponding derivative thereof as the amine component, and an appropriately substituted phosphanylbenzaldehyde or a corresponding ketone as the aldehyde or ketone component.

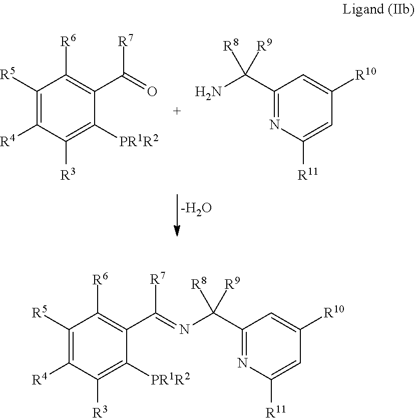

Secondly, it is also possible to use an appropriately substituted phosphanylphenylmethanamine as the amine component and picolinaldehyde or a corresponding derivative thereof as the aldehyde or ketone component.

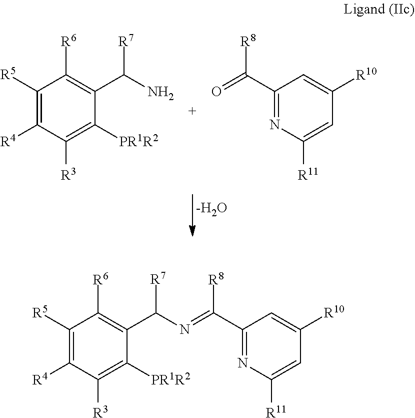

The corresponding starting compounds (amines, ketones or aldehydes) are generally commercially available or can be synthesized using generally known methods. The synthesis of the ligands (IIb) and (IIc) is usually carried out under a protective gas atmosphere. The two components are typically reacted with each another in a solvent at a temperature of 50 to 200° C.

Suitable solvents include, for example, aliphatic alcohols such as methanol, ethanol or isopropanol and aromatic hydrocarbons such as toluene or xylenes. The two starting compounds may be used in stoichiometric amounts. However, it is also possible to use one of the two components in excess, for example in order to increase the conversion of the other component. This is particularly useful if the other component is difficult to access. If an excess is used, the molar ratio of the two starting compounds is generally in the range from >1 to ≤2. The reaction time typically ranges from a few minutes to several hours. Typical reaction times are 10 minutes to 5 hours and preferably 30 minutes to 3 hours. The reaction mixture may be worked up and the ligand isolated by customary methods. However, the solvent and water added are advantageously removed under reduced pressure.

The ligands (IIb) and (IIc) may now be used to prepare the manganese(I) complex (I).

By reducing ligand (IIb) or (IIc) with reducing agents such as sodium borohydride or lithium aluminum hydride or catalytically with hydrogen, ligand (IIa) may be obtained in a simple manner from ligands (IIb) and (IIc). The reaction can be carried out with the common knowledge of those skilled in the art.

In a particularly advantageous synthesis, the condensation described above and the reduction to the ligand (IIa) are carried out directly one after the other in a one-pot reaction without isolating the ligands (IIb) and (IIc) beforehand. To this end, after the condensation has ended, the reducing agent is added directly to the reaction mixture and allowed to react with each other for a further period of time. A few minutes to several hours are usually sufficient here also. Typical reaction times are 10 minutes to 5 hours and preferably 30 minutes to 3 hours. The reaction mixture can then be worked up and the ligand isolated by customary methods. Explicit reference is made to the information given on the work-up and isolation of the ligands (IIb) and (IIc).

Ligand (IIa) may also be formed from ligands (IIb) and (IIc) bonded in the manganese(I) complex (I) under the reaction conditions by hydrogenation with the supplied hydrogen.

The anionic ligand (IId) is formed from the ligand (IIa), as a result of elimination of the hydrogen atom on the nitrogen as a proton, by reaction with a strong base. Suitable strong bases are, for example, NaOMe, NaOEt, KOEt, KOt-Bu or KOMe. Usually, this reaction is not carried out specifically with the free ligand (IIa). Rather, the ligand (IId) may form in the manganese(I) complex (I) under hydrogenation conditions in the presence of a strong base.

The manganese(I) complex to be used in the method according to the invention also bears at least two carbonyl ligands (═CO) in addition to the ligand II.

In the method according to the invention, the oxidation state of the manganese is +1.

The manganese(I) complex (I) preferably used in the method according to the invention has the general formula (I)

[Mn(L)CO)₂₊ₙX₁₋ₙ]Z₍ₙ₎ (1)
where
X is an anionic monodentate ligand having a charge of “−1”
Z is an anionic counterion having a charge of “−1”
n is 0 or 1

The index n indicates whether the manganese(I) complex (I) bears two (n=0) or three CO ligands (n=1). If n=0, the anionic ligand is on the manganese. If n=1, the manganese complex is cationic and the charge is balanced by the anionic counterion Z.

The preferred manganese(I) complex (I) in the method according to the invention is a manganese complex in which
X is an anionic ligand selected from the group of H⁻, F⁻, Cl⁻, Br⁻, I⁻, OH⁻, C₁ to C₆-alkoxy, C₁ to C₆-carboxy, methylallyl, acetylacetonato, RSO₃⁻, CF₃SO₃⁻, CN⁻ and
   BH₄⁻, preferably Br or Cl⁻
Z is an anionic counterion selected from the group of F⁻, Cl⁻, Br⁻, I⁻, OH⁻, C₁ to C₆-alkoxy, C₁ to C₆-carboxy, methylallyl, acetylacetonato, RSO₃⁻, CF₃SO₃⁻, CN⁻, BH₄⁻, BF₄⁻, PF₆⁻, ClO₄⁻, NO₃⁻, BPh₄⁻, preferably Br⁻, Cl⁻, C₁ to C₆-alkoxy and C₁ to C₆-carboxy.

Preferred examples of manganese(I) complexes (I) include [Mn(L)(CO)₂Br], [Mn(L)(CO)₂Cl], [Mn(L)(CO)₂ₗ], [Mn(L)(CO)₂OMe], [Mn(L)(CO)₂CN], [Mn(L)(CO)₂OH], [Mn(L)(CO)₂H], [Mn(L)(CO)₃][Br], [Mn(L)(CO)₃][Cl], [Mn(L)(CO)₃][I], [Mn(L)(CO)₃][OtBu], [Mn(L)(CO)₃][CN], [Mn(L)(CO)₃][NO₃], [Mn(L)(CO)₃][ClO₄], [Mn(L)(CO)₃][BF₄], [Mn(L)(CO)₃][PF₆], where L is in each case the neutral ligands (IIa), (IIb) or (IIc).

The method according to the invention is especially preferably carried out in the presence of manganese(I) complexes (I) in which
the ligand (II) is the ligands (IIa), (IIb) or (IIc) and in which
the radicals R¹, R² are in each case phenyl, p-tolyl, 3,5-dimethyl-4-methoxyphenyl, isobutyl or cyclohexyl,
the radicals R⁵ and R¹⁰ are each independently hydrogen, methyl or tert-butyl,
the radical R¹¹ is each independently hydrogen, methyl or methoxy,
the radicals R⁷, R⁸ and R⁹ are each independently hydrogen or methyl.

Accordingly, in the method according to the invention, the manganese(I) complexes (I) K1, K2, K3, K4 and K5 are particularly suitable.

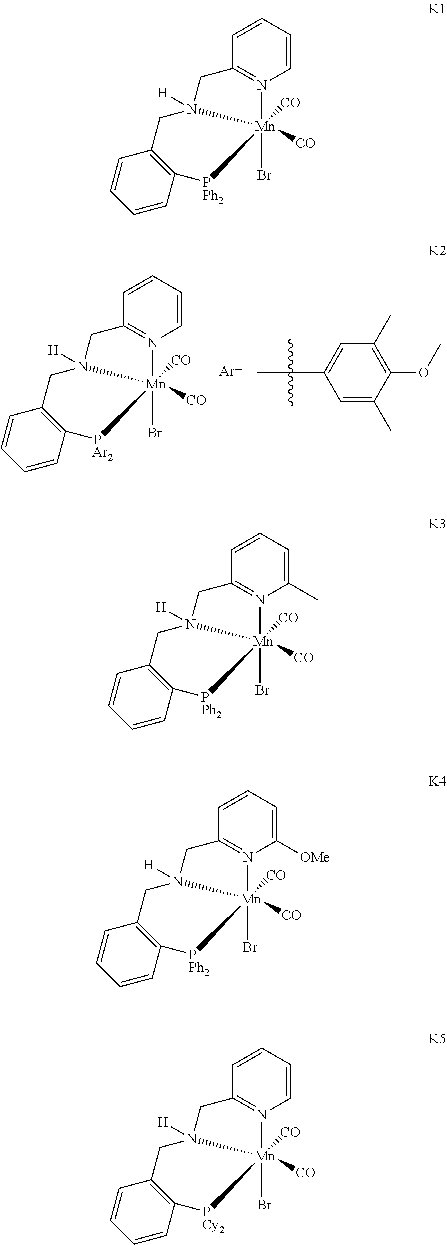

The manganese(I) complexes (I) to be used in the method according to the invention may be obtained in various ways. As manganese-containing starting material, one preferred option is to use a compound in which the manganese is already present in the form of a complex, hereinafter referred to as Mn precursor complex (IV), and to react this with the ligand L. Accordingly, a method is preferred in which the manganese(I) complex (I) is obtained by reacting ligand (II) with a Mn precursor complex (IV).

In principle, a very wide variety of Mn complexes may be used as Mn precursor complex (IV). In many cases it is not necessary that the Mn precursor complex (IV) already comprises the ligands X or carbonyl and optionally the non-coordinating anion Z of the desired manganese complex (I). The ligands X, CO and the non-coordinating anion Z may also in many cases be added separately to the synthesis batch. In order to keep the synthetic effortlow, readily accessible or readily available complexes are advantageously used as Mn precursor complexes (IV). Such complexes are well known to those skilled in the art. The person skilled in the art is also familiar with the exchange of ligands on manganese-containing complexes.

In principle, all anionic ligands which have already been described under ligand X are eligible as anionic ligands in the Mn precursor complex (IV).

The reaction of the Mn precursor complex (IV) with the ligand L is typically carried out at a Mn/L molar ratio from 0.8 to 20, preferably from 0.9 to 10 and particularly preferably from 0.9 to 1.1. In order to achieve the highest possible degree of conversion, it is advantageous to use a Mn precursor complex (IV) having only monodentate and bidentate ligands in order to utilize the complexing effect of the tridentate ligand L. The reaction is usually carried out anhydrously, but in the presence of a solvent and under a protective gas atmosphere. Suitable solvents include, for example, aliphatic alcohols such as methanol, ethanol or isopropanol and aromatic hydrocarbons such as toluene or xylenes. In general, the manganese in the Mn precursor complex (IV) has the same oxidation state as in the subsequent manganese(I) complex (I), i.e. the oxidation state+I.

Suitable Mn precursor complexes (IV) include, for example, [Mn(CO)₅Br], [Mn(CO)₅Cl], [Mn(CO)₅ₗ], [Mn(CO)₅F], [Mn₂(CO)₁₀], MnCl₂, Mn(OAc)₂, Mn(OAc)₃, Mn(acac)₂, Mn(acac)₃, MnBr2, Mnl2, MnCO₃, Mn(NO₃)₂ and Mn(ClO₄)₂. (acac=acetylacetonate)

The manganese(I) complex (I) may be isolated from the reaction mixture obtained, for example by precipitation or crystallization.

To carry out the hydrogenation according to the invention, however, it is generally not necessary to first isolate the manganese(I) complex (I) after its preparation. Rather, it is advantageous in the sense of a simplified procedure to prepare the manganese(I) complex (I) as described above from a Mn precursor complex (IV) and the ligand L in the presence of a solvent and to carry out the hydrogenation according to the invention directly in the reaction mixture obtained.

In the method according to the invention, different manganese(I) complexes (I), in particular also mixtures of cationic and neutral manganese(I) complexes (I) containing the same ligand L (II), may also be used.

The esters to be used in the method according to the invention can be of a diverse nature. Thus, in principle, linear or branched, non-cyclic or cyclic, saturated or unsaturated, aliphatic, aromatic or araliphatic esters, that are unsubstituted or interrupted by heteroatoms or functional groups, of different molar masses from low to high molecular weight may be used.

The ester used is preferably an ester of the general formula (III)

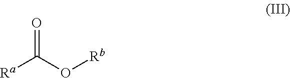

in which the radicals R^{a} and R^{b} are each independently a carbon-containing organic, linear or branched, non-cyclic or cyclic, saturated or unsaturated, aliphatic, aromatic or araliphatic radical which is unsubstituted or interrupted or substituted by heteroatoms or functional groups and has a molar mass of 15 to 10 000 g/mol, it also being possible for the two radicals R^{a} and R^{b} to be bonded to each other.

In the case of branched radicals R^{a} and R^{b}, these may be branched one or multiple times. Likewise, in the case of cyclic radicals, these may be monocyclic or polycyclic. Likewise, in the case of unsaturated radicals, these may be mono- or polyunsaturated, both double bonds and triple bonds being possible here. Heteroatoms are to be understood as atoms that are neither carbon nor hydrogen. Preferred examples of heteroatoms include oxygen, nitrogen, sulfur, phosphorus, fluorine, chlorine, bromine and iodine, and particularly preferred examples are oxygen, nitrogen, fluorine, chlorine and bromine. Functional groups are another description of groups comprising at least one heteroatom. For example, a hydrocarbon chain interrupted by —O— can be considered both as a hydrocarbon chain interrupted by an oxygen heteroatom and as a hydrocarbon chain interrupted by an ether group. Other non-limiting examples include amino groups (—NH₂, —NH—, —N<), aldehyde groups (—CHO), carboxyl groups (—COOH), amide groups (—CONH₂, —CONH—, —CON<), nitrile groups (—CN), isonitrile groups (—NC), nitro groups (—NO₂), sulfonic acid groups (—SO₃), keto groups (>CO), imino groups (>CNH, >CN—), ester groups (—CO—O—), anhydride groups (—CO—O—CO—) and imido groups (—CO—NH—CO—, —CO—NR—CO—). Of course, also two or more so-called functional groups may be present. An example here includes fats.

If the radicals R^{a} and R^{b} are bonded to each other, these are cyclic esters, which are also referred to as lactones.

The molar masses of the radicals R^{a} and R^{b} are generally 15 to 10 000 g/mol, preferably 15 to 5000 g/mol and particularly preferably 15 to 2000 g/mol.

In the method according to the invention, preference is given to using esters having a molar mass of 74 to 20 000 g/mol, particularly preferably 74 to 10 000 g/mol, very particularly preferably 74 to 5000 g/mol, especially 74 to 2000 g/mol and in particular 74 to 1000 g/mol.

The molecular hydrogen (H₂) to be used in the method according to the invention can be supplied either undiluted or diluted with inert gas, for example nitrogen. It is advantageous to supply a hydrogen-containing gas with the highest possible hydrogen content. Preference is given to a hydrogen content of ≥80% by volume, particularly preferably of ≥90% by volume, especially preferably of ≥95% by volume and particularly of ≥99% by volume.

In a very general embodiment of the method according to the invention, the manganese(I) complex (I), the ester to be hydrogenated and hydrogen are fed to a suitable reaction apparatus and the mixture is reacted under the desired reaction conditions.

The reaction apparatus used in the method according to the invention may in principle be any reaction apparatus which are suitable in principle for gas/liquid reactions under the specified temperature and the specified pressure. Suitable standard reactors for gas/liquid and for liquid/liquid reaction systems are described, for example, in K. D. Henkel, “Reactor Types and Their Industrial Applications”, in Ullmann's Encyclopedia of Industrial Chemistry, 2005, Wiley-VCH Verlag GmbH & Co. KGaA, DOI: 10.1002/14356007.b04_087, Chapter 3.3 “Reactors for gas-liquid reactions”. Examples include stirred tank reactors, tubular reactors or bubble column reactors. Pressure-resistant stirred tanks are usually also referred to as autoclaves.

The manganese(I) complex (I) can be fed directly to the reaction apparatus in the form of the previously synthesized manganese(I) complex (I).

It is much simpler to form the manganese(I) complex (I) in situ from an Mn precursor complex (IV) and ligand L (II). In situ means that the manganese(I) complex (I) is formed by supplying Mn precursor complex (IV) and ligand L (II) to the reaction apparatus. Advantageously for this purpose, a molar ratio of ligand L (II) to manganese of 0.5 to 5, preferably ≥0.8 and particularly preferably ≥1, and preferably ≤3, particularly preferably ≤2 an d especially preferably ≤1.5 is used. This in situ variant spares the prior isolation of the manganese(I) complex (I).

The method according to the invention may be carried out in the presence or also in the absence of a solvent. If a solvent is used, this serves, for example, to dissolve the manganese(I) complex (I) or a Mn precursor complex (IV) and the ligand L, but also optionally to dissolve the ester to be hydrogenated. Especially in the case of low molecular weight esters, said esters may also function as solvent.

If solvents are used, solvents having more or less pronounced polar properties which are not themselves hydrogenated under the reaction conditions are preferred. Preferred examples include aliphatic alcohols such as methanol, ethanol or isopropanol and aromatic hydrocarbons such as toluene or xylenes and ethers such as tetrahydrofuran or 1,4-dioxane. The amount of solvent used can vary widely. However, amounts in the range from 0.1 to 20 g of solvent per g of ester to be hydrogenated, preferably 0.5 to 10 g of solvent per g of ester to be hydrogenated and particularly preferably 1 to 5 g of solvent per g of ester to be hydrogenated, are customary.

The ester to be hydrogenated may be supplied directly in the form of the pure, undiluted ester, but also diluted or dissolved in a solvent. The criteria in which form the ester to be hydrogenated is added are generally often of a purely practical nature, such as, for example, the nature of the ester present and its handling. For example, the aim is for the ester in the reaction mixture to be in liquid form under the reaction conditions.

The molar ratio between the ester to be hydrogenated and the manganese (I) complex (I) may vary within a wide range in the method according to the invention. In general, the molar ratio specified in the reaction mixture to be hydrogenated is from 1 to 100 000, preferably from 10 to 25 000, particularly preferably from 100 to 5000 and especially preferably from 500 to 20 000.

The method according to the invention is carried out at a temperature of 50 to 200° C., preferably at ≤170° C. and particularly preferably at ≤150° C. The pressure in this case is 0.1 to 20 MPa abs, preferably ≥1 MPa abs and particularly preferably ≥5 MPa abs, and preferably ≤15 MPa abs and particularly preferably ≤10 MPa abs.

The reaction time or mean residence time in which the reaction mixture is present under the reaction conditions can also vary widely, but is typically in the range from 0.1 to 100 hours, preferably ≥1 hour and particularly preferably ≥2 hours, and preferably ≤80 hours and particularly preferably ≤60 hours.

Furthermore, it has been shown that the hydrogenation according to the invention is generally positively influenced by the presence of a base and, as a result, significantly higher conversions are ultimately made possible. Therefore, in most cases it is advantageous to carry out the hydrogenation in the presence of a base. In principle, the bases may also be present in the reaction mixture as a solid, but bases which are present in dissolved form in the reaction mixture are preferred. Examples of possible bases include alkoxides, hydroxides, alkali metal and alkaline earth metal carbonates, amides, basic aluminum and silicon compounds and also hydrides. The bases used are particularly preferably alkoxides or amides, preferably sodium methoxide, potassium methoxide, sodium hydroxide, sodium ethoxide, potassium ethoxide, potassium tertbutoxide, sodium tert-butoxide, sodium borohydride or sodium hydride.

If the method according to the invention is carried out in the presence of a base, this is generally used in excess with respect to the manganese(I) complex (I). Preference is given to using a molar ratio of base to manganese complex (I) of 1 to 1000, preferably of 2 to 20, particularly preferably of 1 to 10.

The method according to the invention may be carried out continuously, in semi-batch mode, discontinuously, back-mixed in the product as solvent or in a single pass not back-mixed. The manganese(I) complex, the ester to be hydrogenated, the hydrogen, optionally the solvent and optionally the base can be fed in simultaneously or separately from one another.

In the discontinuous mode of operation, the manganese(I)complex (I) or an Mn precursor complex (IV) and the ligand L (II), the ester to be hydrogenated and optionally solvent and a base are typically initially charged in the reaction apparatus and the desired reaction pressure under the desired reaction conditions is set with mixing by addition of hydrogen. The reaction mixture is then left under the desired reaction conditions for the desired reaction time. Optionally, additional hydrogen is metered in. After the desired reaction time has elapsed, the reaction mixture is cooled or depressurized. The corresponding alcohols can be obtained as reaction products by subsequent work-up. The discontinuous reaction is preferably carried out in a stirred tank.

In the continuous mode of operation, the manganese(I) complex (I) or a Mn precursor complex (IV) and the ligand L (II), the ester to be hydrogenated and optionally solvent and a base are continuously fed to the reaction apparatus and an appropriate amount is continuously withdrawn for work-up and isolation of the corresponding alcohol formed.

The continuous reaction is preferably carried out in a stirred tank or a stirred tank cascade.

The hydrogenation product may be separated from the hydrogenation mixture by processes known per se to those skilled in the art such as by distillation and/or flash evaporation, and the remaining catalyst may be utilized in the context of further reactions. In the context of the preferred embodiment it is advantageous to omit addition of solvents and to carry out the reactions cited in the substrate to be converted or in the product and optionally in high-boiling by-products as the dissolution medium. Particular preference is given to the continuous reaction regime with reuse or recycling of the homogeneous catalyst.

In the ester hydrogenation according to the invention, a terminal —CH₂OH and a terminal —OH group are formed from the —CO—O— ester group. In the case of the esters (III), therefore, the two corresponding alcohols R^{a}—CH₂OH and R^{b}—OH are formed corresponding to the following reaction equation.

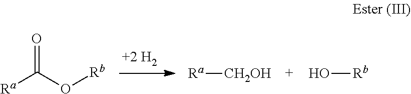

When using cyclic esters, so-called lactones, the two radicals R^{a} and R^{b} are bonded to each other and the corresponding diol is formed.

The method according to the invention enables the preparation of alcohols in high yield and selectivity by homogeneously catalyzed hydrogenation of esters. The hydrogenation may be carried out technically in conventional laboratory equipment for hydrogenation reactions and enables the use of a wide variety of esters as substrates.

A further embodiment of the invention is the conversion of sclareolide to ambroxidol (ambrox-1,4-diol), which is a precursor to the important fragrance (−) Ambrox. Ambroxidol can be converted to (−) Ambrox by cyclization, as described for example in WO 2017/140909.

The particular advantages of the method according to the invention are based on the specific, tridentate PNN ligands. Due to its tridentate nature, the ligand coordinates tightly to the manganese, but is easy to prepare and affords catalysts with high hydrogenation activities after formation of corresponding manganese(I) complexes. In addition, the ligand according to the invention is also relatively insensitive to oxidation, so that it is also advantageous in terms of handling and has high storage stability.

The particular advantages of the ligand according to the invention most notably include its ready accessibility and easy possibility of varying the basic structure by replacing hydrogen atoms with various organic radicals. The ligand can generally be prepared fro m readily available feedstocks by a simple one-pot synthesis. Manganese precursor complexes that are readily accessible and commercially available in large quantities may be used as manganese-containing feedstocks.

### EXAMPLES

### General Information

Unless otherwise stated, all reactions were prepared at room temperature under an argon atmosphere using so-called “Schlenk” and high vacuum techniques or in an MBraun Inert Atmosphere glove box. Organic solvents were sourced from Aldrich or Acros. Commercially available starting compounds were sourced from Aldrich, ABCR or TCI and used as received. NMR spectra were measured on Bruker AVANCE III 300, Bruker AVANCE III 400 and Bruker AVANCE III 500 spectrometers and the protons (¹H) or carbon (¹³C) resonance signals of the solvent served as reference. Chemical shifts (δ) are expressed in ppm. ³¹P-NMR spectra refer to an external standard (ampoule D₃PO₄) of the Organic Chemistry Institute of the University of Heidelberg. GC analyzes were carried out on an Agilent Technologies 6890N gas chromatograph equipped with an FID detector; column used: DB-FFAP (30 m×0.32 mm×0.25 μm). Initial temperature: 55° C.; hold time 1 min; ramp: 25° C./min to 250° C.; hold time: 8 min.

In Example 1, the preparation of a representative ligand II is described. The other ligands were prepared analogously to this specification.

### Example 1: Preparation of Ligand L1

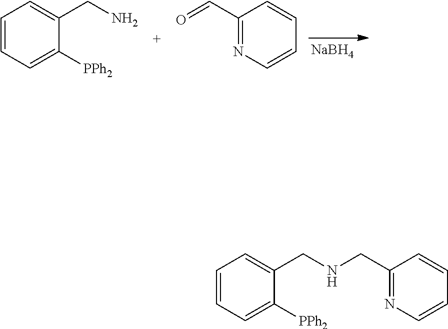

(2-(Diphenylphosphaneyl)phenyl)methanamine (1.00 g, 3.43 mmol) was added at room temperature to a solution of picolinaldehyde (368 mg, 3.43 mmol) in ethanol (10 mL) and the resulting mixture stirred at room temperature for 2 hours. NaBH₄ (208 mg, 5.49 mmol) was added and the mixture was stirred at room temperature for a further 2 hours. Aqueous saturated NaHCO₃ solution (15 ml) and CH₂Cl₂ (25 mL) were then added. After phase separation, the aqueous phase was extracted with CH₂Cl₂ (2×25 ml). The combined organic phase was dried (Na₂SO₄) and concentrated in vacuo. The crude product was purified by column chromatography on silica gel (hexane/EtOAc/NEt₃, 9:1 to 1:1; a mixture of 10% NEt₃ in EtOAc was used) and N-(2-(diphenylphosphaneyl)benzyl)-1-(pyridin-2-yl) methanamine (1) was obtained as a colorless oil (600 mg, 46% yield).

¹H NMR (500 MHz, CD₂Cl₂) δ 8.48-8.46 (m, 1H), 7.57 (td, J=7.7, 1.8 Hz, 1H), 7.54-7.51 (m, 1H), 7.36-7.30 (m, 7H), 7.28-7.24 (m, 4H), 7.19-7.10 (m, 4H), 6.91 (ddd, J=7.7, 4.5, 1.4 Hz, 1H), 4.02 (d, J=1.7 Hz, 2H), 3.79 (s, 2H). ³¹P NMR (203 MHz, CD₂Cl₂) δ-15.94.

HRMS (ESI) C₂₅H₂₃N₂P ([M]⁺): calculated: 382.1599; found: 382.1611.

In Example 2, the preparation of a representative manganese(I) catalyst complex (I) with a ligand II is described.

### Example 2: Preparation of Catalyst K1

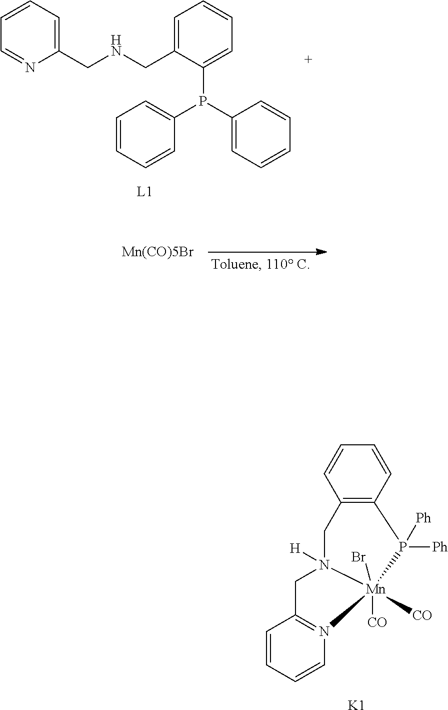

A solution of [Mn(CO)₅Br] (720 mg, 2.62 mmol) in 15 ml of toluene are added to a solution of the ligand L1 (1 g, 2.62 mmol) in 15 ml of toluene under an argon atmosphere. The reaction mixture is stirred at 60-80° C. for 30 minutes until no visible CO evolution can be observed. The mixture is then stirred for a further 16 hours at 110° C., producing a yellow precipitate. After cooling to room temperature, the toluene is removed from the reaction mixture under reduced pressure. The yellow residue is washed with 10 ml of absolute hexane and then with 10 ml of absolute diethyl ether under an argon atmosphere and filtered off. The filter cake is dried in a high vacuum and the catalyst K1 is obtained as a yellow powder at a yield of 80% (1.2 g). The catalyst complex is stored under exclusion of light at 0° C.

¹H NMR (500 MHz, CD₂Cl₂) δ 9.09 (d, J=5.5 Hz, 1H), 8.07 (s, 2H), 7.67 (t, J=7.1 Hz, 1H), 7.57-7.22 (m, 11H), 7.05 (t, J=8.5 Hz, 2H), 6.81 (t, J=8.2 Hz, 1H), 4.59-3.59 (m, 5H). ¹³C NMR (126 MHz, CD₂Cl₂) δ 154.62, 151.77, 135.76 (d, J=16.5 Hz), 133.55-133.09 (m), 129.76, 129.56, 129.00, 128.79 (d, J=3.2 Hz), 128.70, 127.93 (d, J=8.4 Hz), 127.18 (dd, J=7.9, 2.2 Hz), 126.13 (d, J=5.2 Hz), 125.92 (d, J=2.0 Hz), 124.93 (d, J=9.0 Hz), 124.63 (d, J=9.5 Hz), 120.93, 117.01, 56.86 (d, J=2.6 Hz), 55.71 (d, J=8.1 Hz). ³¹P-NMR (203 MHz, CD₂Cl₂) δ 68.39.

### Example 3: Preparation of Catalyst K2

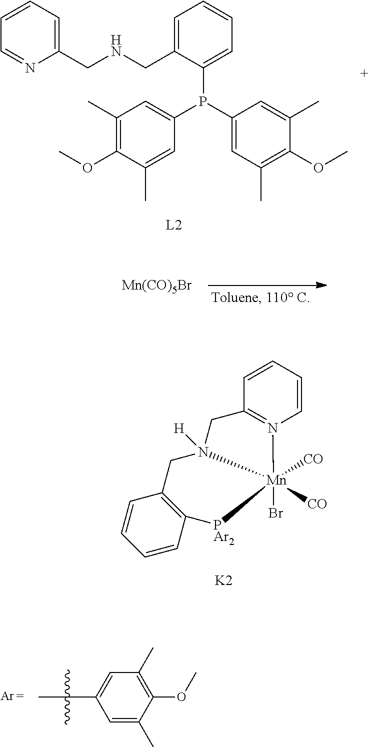

A solution of [Mn(CO)₅Br] (85 mg, 0.31 mmol) in 10 ml of toluene are added to a solution of the ligand L2 (0,155 g, 0.31 mmol) in 10 ml of toluene under an argon atmosphere. The reaction mixture is stirred at 60-80° C. for 30 minutes until no visible CO evolution can be observed. The mixture is then stirred fora further 16 hours at 110° C., producing a yellow precipitate. After cooling to room temperature, the toluene is removed from the reaction mixture under reduced pressure. The yellow residue is washed with 10 ml of absolute hexane and then with 10 ml of absolute diethyl ether under an argon atmosphere and filtered off. The filter cake is dried in a high vacuum and the catalyst K2 is obtained as a yellow powder at a yield of 75% (0.16 g). The catalyst complex is stored under exclusion of light at 0° C.

### Example 4: Preparation of Catalyst K3

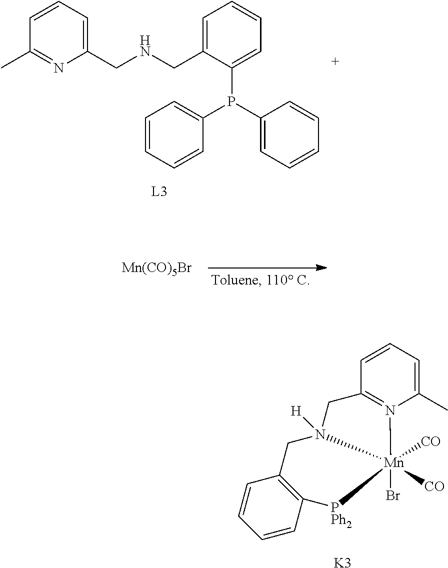

A solution of [Mn(CO)₅Br] (97 mg, 0.35 mmol) in 10 ml of toluene are added to a solution of the ligand L3 (0.14 g, 0.35 mmol) in 10 ml of toluene under an argon atmosphere. The reaction mixture is stirred at 60-80° C. for 30 minutes until no visible CO evolution can be observed. The mixture is then stirred for a further 16 hours at 110° C., producing a yellow precipitate. After cooling to room temperature, the toluene is removed from the reaction mixture under reduced pressure. The yellow residue is washed with 10 ml of absolute hexane and then with 10 ml of absolute diethyl ether under an argon atmosphere and filtered off. The filter cake is dried in a high vacuum and the catalyst K3 is obtained as a yellow powder at a yield of 56% (0,115 g). The catalyst complex is stored under exclusion of light at 0° C.

### Example 5: Preparation of Catalyst K4

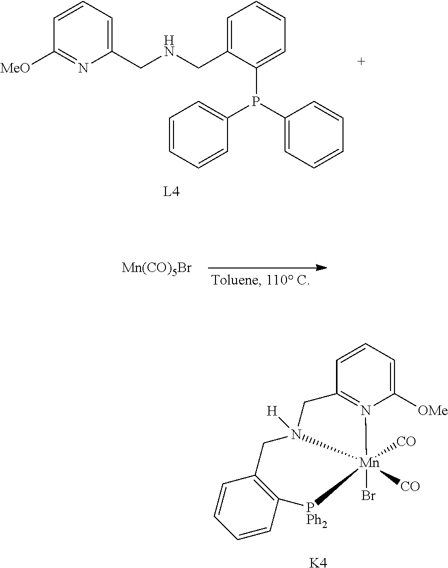

A solution of [Mn(CO)₅Br] (0.36 g, 1.31 mmol) in 10 ml of toluene are added to a solution of the ligand L4 (0.54 g, 1.31 mmol) in 10 ml of toluene under an argon atmosphere. The reaction mixture is stirred at 60-80° C. for 30 minutes until no visible CO evolution can be observed. The mixture is then stirred for a further 16 hours at 110° C., producing a yellow precipitate. After cooling to room temperature, the toluene is removed from the reaction mixture under reduced pressure. The yellow residue is washed with 10 ml of absolute hexane and then with 10 ml of absolute diethyl ether under an argon atmosphere and filtered off. The filter cake is dried in a high vacuum and the catalyst K4 is obtained as a yellow powder at a yield of 82% (0.65 g). The catalyst complex is stored under exclusion of light at 0° C.

### Example 5: Preparation of Catalyst K5

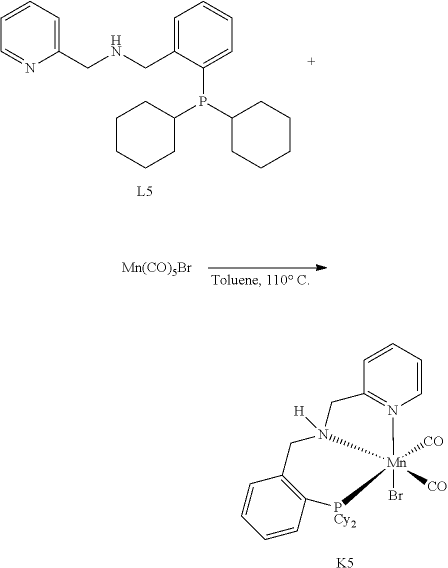

A solution of [Mn(CO)₅Br] (0,236 g, 0.86 mmol) in 10 ml of toluene are added to a solution of the ligand L5 (0.34 g, 0.86 mmol) in 10 ml of toluene under an argon atmosphere. The reaction mixture is stirred at 60-80° C. for 30 minutes until no visible CO evolution can be observed. The mixture is then stirred for a further 16 hours at 110° C., producing a yellow precipitate. After cooling to room temperature, the toluene is removed from the reaction mixture under reduced pressure. The yellow residue is washed with 10 ml of absolute hexane and then with 10 ml of absolute diethyl ether under an argon atmosphere and filtered off. The filter cake is dried in a high vacuum and the catalyst K5 is obtained as a yellow powder at a yield of 75% (0.38 g). The catalyst complex is stored under exclusion of light at 0° C.

### Examples 6, 7, 8 and 9: Hydrogenation of Sclareolide

In an argon-filled glove box, sclareolide (375 mg, 1.5 mmol), manganese catalyst (0.1 mol %), KOtBu (3.36 mg, 2 mol %) and dry ethanol (2 ml) are filled into 10 mL vials with crimp caps and PTFE-coated magnetic stirrer bars. The vials are sealed with the crimp cap comprising a rubber septum, the septum is pierced with a cannula and the vials are placed in a HEL CAT-7 autoclave. The autoclave is sealed, removed from the glove box and pressurized to 50 bar H₂ under inert conditions, and the autoclave is inserted into a preheated aluminum block. The reaction mixture is stirred at 100° C. for 20 h, cooled in an ice bath and the remaining H₂ pressure is carefully released. Mesitylene is then added to the respective batches as internal standard and the reaction mixtures are analyzed by gas chromatography.

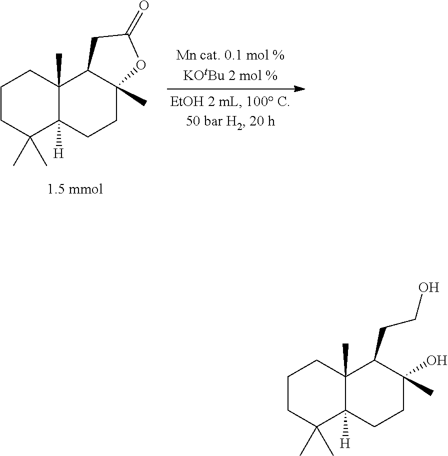 [SMILES: CC1(C)CCC[C@]2(C)[C@H]3CC(=O)O[C@]3(C)CC[C@@H]12 ; CC1(C)CCC[C@]2(C)[C@@H](CCO)[C@](C)(O)CC[C@@H]12]

| Example | Catalyst | Conversion | GC yield of diol |
|---|---|---|---|
| 6 | K1 | full | 93% |
| 7 | K2 | full | 91.5% |
| 8 | K4 | 34% | 34% |
| 9 | K5 | full | 94% |

### Example 10: Hydrogenation of Sclareolide

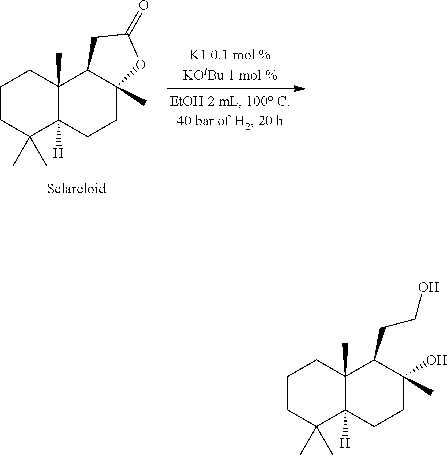 [SMILES: CC1(C)CCC[C@]2(C)[C@H]3CC(=O)O[C@]3(C)CC[C@@H]12 ; CC1(C)CCC[C@]2(C)[C@@H](CCO)[C@](C)(O)CC[C@@H]12]

In an argon-filled glove box under inert conditions, sclareolide (750 mg, 3 mmol), K1 (1.7 mg, 0.003 mmol), potassium tert-butoxide (3.36 mg, 0.003 mmol) and 4 ml of dry ethanol are weighed into a 100 ml Premex autoclave with Teflon insert and magnetic stirrer bar. The autoclave is sealed and evacuated. It is then flushed three times with nitrogen and then three times with hydrogen and pressurized with hydrogen to 40 bar cold pressure. The reaction mixture is then heated in the autoclave at 90° C. with stirring for 20 h. After cooling to room temperature, the remaining hydrogen is released and the reaction mixture is analyzed by GC. Sclareolide conversion: 98%, yield of diol: 92%.

### (2R, 8aS)-1-(2-hydroxyethyl)-2,5,5,8a-tetramethyldecahydronaphthalen-2-ol

¹H NMR (400 MHz, CDCl₃) δ 3.78 (dt, J=10.2, 4.4 Hz, 1H), 3.46 (ddd, J=10.2, 8.2, 5.7 Hz, 1H), 3.08 (s, 2H), 1.90 (dt, J=12.3, 3.3 Hz, 1H), 1.72-1.21 (m, 10H), 1.19 (s, 3H), 1.13 (dd, J=13.4, 4.4 Hz, 1H), 0.99-0.91 (m, 2H), 0.88 (s, 3H), 0.79 (s, 6H).

¹³C-NMR (101 MHz, CDCl₃) δ 73.03, 64.09, 59.18, 56.04, 44.29, 41.91, 39.36, 38.98, 33.41, 33.28, 27.89, 24.64, 21.48, 20.47, 18.42, 15.31.

### Example 11: Hydrogenation of Sclareolide

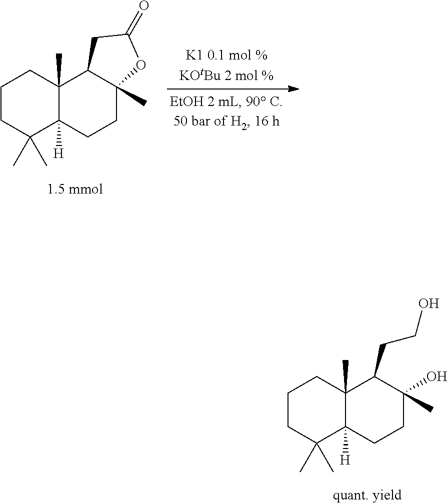 [SMILES: CC1(C)CCC[C@]2(C)[C@H]3CC(=O)O[C@]3(C)CC[C@@H]12 ; CC1(C)CCC[C@]2(C)[C@@H](CCO)[C@](C)(O)CC[C@@H]12]

In an argon-filled glove box, sclareolide (375 mg, 1.5 mmol), manganese catalyst K1 (0.1 mol %), KOtBu (3.36 mg, 2 mol %) and dry ethanol (2 ml) are filled into a 10 ml vial with crimp cap and PTFE-coated magnetic stirrer bar. The vials are sealed with the crimp cap comprising a rubber septum, the septum is pierced with a cannula and the vials are placed in a HEL CAT-7 autoclave. The autoclave is sealed, removed from the glove box and pressurized to 50 bar H₂, and the autoclave is inserted into a preheated aluminum block. The reaction mixture is stirred at 90° C. for 16 h, cooled in an ice bath and the remaining H₂ pressure is carefully released. The reaction mixture is filtered over silica and the silica is washed several times with ethanol. The ethanol is removed from the combined filtrates in vacuo to dryness and the product is analyzed by NMR. The isolated yield of sclareolide diol is quantitative (yield>99%) and the substance is pure according to the 1H-NMR spectrum.

### Example 12: Hydrogenation of Sclareolide

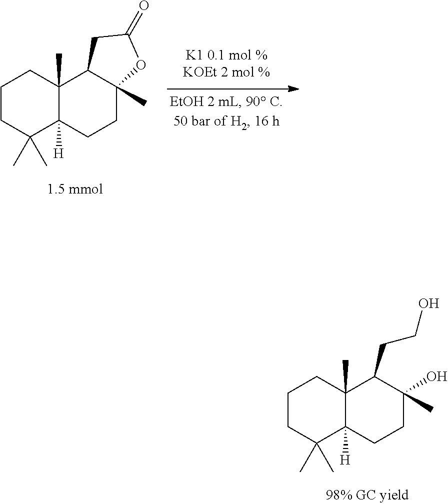 [SMILES: CC1(C)CCC[C@]2(C)[C@H]3CC(=O)O[C@]3(C)CC[C@@H]12 ; CC1(C)CCC[C@]2(C)[C@@H](CCO)[C@](C)(O)CC[C@@H]12]

In an argon-filled glove box under inert conditions, sclareolide (375 mg, 1.5 mmol), K1 (0.88 mg, 0.1 mol %), potassium ethoxide (2.52 mg, 2 mol %) and 2 ml of dry ethanol are weighed into a 30 ml Premex autoclave with Teflon insert and magnetic stirrer bar. The autoclave is sealed and evacuated. It is then flushed three times with nitrogen and then three times with hydrogen and pressurized with hydrogen to 40 bar cold pressure. The reaction mixture is then heated in the autoclave at 90° C. with stirring for 16 h. After cooling to room temperature, the remaining hydrogen is released and the reaction mixture is analyzed by GC. Sclareolide conversion: >99%, yield of diol: 98%.

Examples 11, 12, 13, 14, and 15: Hydrogenation of other esters with the catalyst K1 In an argon-filled glove box, the respective ester (3 mmol), manganese catalyst K1 (1.72 mg, 0.1 mol %), KOtBu (6.72 mg, 2 mol %) and dry ethanol (4 ml) are filled into 10 mL vials with crimp caps and PTFE-coated magnetic stirrer bars. The vials are sealed with the crimp cap comprising a rubber septum, the septum is pierced with a cannula and the vials are placed in a HEL CAT-7 autoclave. The autoclave is sealed, removed from the glove box and pressurized to 50 bar H₂, and the autoclave is inserted into a preheated aluminum block. The reaction mixture is stirred at 100° C. for 20 h, cooled in an ice bath and the remaining H₂ pressure is carefully released. The reaction mixtures are filtered over silica and the silica is washed several times with ethanol. The ethanol is removed from the combined filtrates in vacuo to dryness and the product is analyzed by NMR.

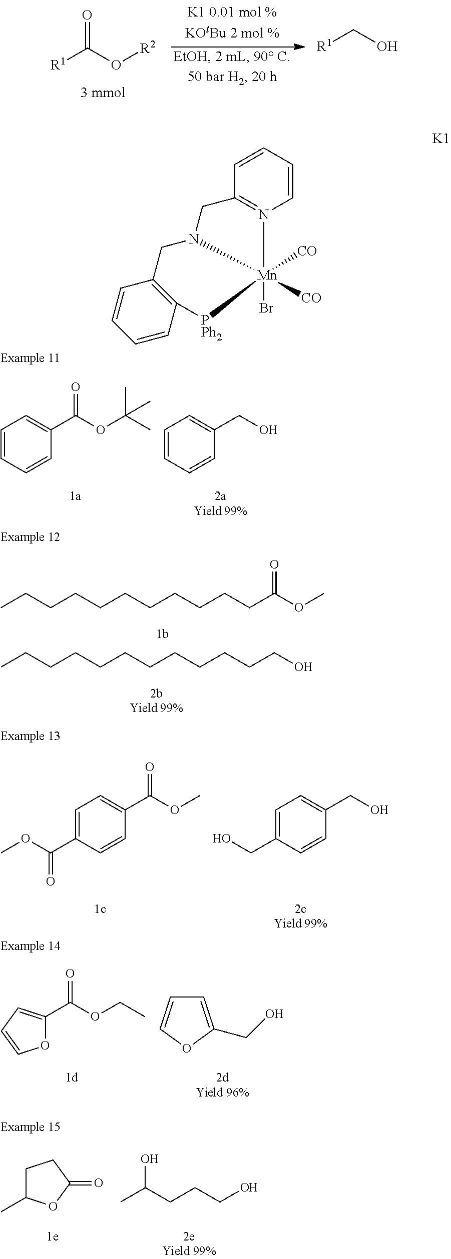

### Phenylmethanol, 2a

¹H NMR (301 MHz, CDCl₃) δ 7.39-7.26 (m, 5H), 4.66 (s, 2H), 1.98 (s, 1H). ¹³C NMR (76 MHz, CDCl₃) δ 140.89, 128.58, 127.66, 127.02, 65.34.

### Dodecan-1-ol, 2b

¹H NMR (301 MHz, CDCl₃) δ 3.64 (t, J=6.6 Hz, 2H), 1.55 (q, J=7.1 Hz, 2H), 1.37-1.26 (m, 18H), 0.93-0.83 (m, 3H).

¹³C NMR (76 MHz, CDCl₃) δ 63.09, 32.82, 31.92, 29.67, 29.64, 29.62, 29.61, 29.45, 29.35, 25.75, 22.69, 14.11.

### 1,4-phenylenedimethanol, 2c

¹H NMR (301 MHz, CDCl₃) δ 7.37 (s, 4H), 4.70 (s, 4H), 1.64 (s, 2H). ¹³C NMR (76 MHz, CDCl₃) δ 127.25, 88.96.

### Furan-2-ylmethanol, 2d

¹H NMR (301 MHz, CDCl₃) δ 7.39 (s, 1H), 6.31 (d, J=15.0 Hz, 2H), 4.58 (s, 2H), 2.27 (s, 1H).

¹³C NMR (76 MHz, CDCl₃) δ 154.03, 142.57, 110.36, 107.76, 57.42.

### Pentane-1,4-diol, 2e

¹H NMR (301 MHz, CDCl₃) δ 3.94-3.78 (m, 1H), 3.77-3.58 (m, 2H), 2.72 (s, 2H), 1.76-1.39 (m, 4H), 1.21 (d, J=6.2 Hz, 3H).

¹³C NMR (76 MHz, CDCl₃) δ 67.95, 62.89, 36.26, 29.14, 23.60.

1.-6. (canceled)

7. A method for hydrogenating an ester of the general formula (III) 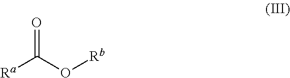
in which the radicals R^{a} and R^{b} are each independently a carbon-containing organic, linear or branched, non-cyclic or cyclic, saturated or unsaturated, aliphatic, aromatic or araliphatic radical which is unsubstituted or interrupted or substituted by heteroatoms or functional groups and has a molar mass of 15 to 10 000 g/mol, wherein the two radicals R^{a} and R^{b} may also be bonded to each other,
with molecular hydrogen to give the alcohols 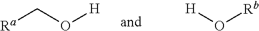
at a temperature of 50 to 200° C. and a pressure of 0.1 to 20 MPa abs in the presence of a manganese(I) complex, in which the manganese complex comprises a tridentate ligand L of the general formula (II) 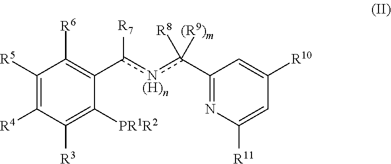
and comprises at least two carbonyl ligands, wherein
R¹, R² are each independently an aliphatic hydrocarbon radical having 1 to 8 carbon atoms, an aromatic hydrocarbon radical having 6 or 10 carbon atoms or an araliphatic hydrocarbon radical having 7 to 12 carbon atoms, where the hydrocarbon radicals specified are unsubstituted or substituted by 1 to 3 methoxy, thiomethoxy or dimethylamino groups, and the two radicals R¹ and R² may be bonded to each other to form a 5- to 10-membered ring including the phosphorus atom,
R³, R⁴, R⁵, R⁶, R¹⁰, R¹¹ are each independently hydrogen, linear C₁ to C₄-alkyl, branched C₃ to C₄-alkyl, methoxy, hydroxyl, trifluoromethyl, nitrile or dialkylamino each independently having 1 to 4 carbon atoms per alkyl group,
R⁷, R⁸, R⁹ are each independently hydrogen, linear C₁ to C₄-alkyl or branched C₃ to C₄-alkyl, n, m are each independently 0 or 1, and the solid-dashed double lines are a single or double bond, with the proviso that
in the case of n=1, both solid-dashed double lines are a single bond and m is 1, and
in the case of n=0, one solid-dashed double line is a single bond and the other solid-dashed double line is a double bond,
wherein, in the case of a double bond on the side facing the phenyl ring m=1, in the case of a double bond on the side
facing the pyridyl ring m=0, or both solid-dashed double lines are a single bond and m
equals 1.

8. The method according to claim 7, wherein the manganese complex has a general formula (I)
[Mn(L)(CO)₂₊ₙX₁₋ₙ]Z₍ₙ₎ (I)
wherein
X is an anionic monodentate ligand having a charge of “−1”
Z is an anionic counterion having a charge of “−1”
N is 0 or 1.

9. The method according to claim 7, wherein a molar ratio between the ester and the manganese(I) complex I is from 100 to 100 000.

10. The method according to claim 7, wherein the reaction is carried out in the presence of a base as co-catalyst.

11. The method according to claim 7, wherein the ester III is sclareolide, which is hydrogenated to the corresponding diol.

12. A method for producing (−) Ambrox, comprising hydrogenating sclareolide to Ambrox-1,4-diol in a first step (i) according to claim 11, and the resulting Ambrox-1,4-diol is cyclized to (−) Ambrox in a second step (ii).
